(19) United States
(12) Patent Application Publication
(10) Pub. No.: US 2023/0204573 A1
(43) Pub. Date: Jun. 29, 2023

(54) NANOPHOTODETECTOR-BASED DEVICE FOR BIOMOLECULAR DETECTION

(71) Applicants: Commissariat à l'Energie Atomique et aux Energies Alternatives, Paris (FR); Centre National de la Recherche Scientifique, Paris (FR)

(72) Inventors: Olivier CONSTANTIN, Grenoble Cedex 9 (FR); Moira HOCEVAR, Grenoble (FR); Ali JAFFAL, Grenoble Cedex 9 (FR); Pascal MAILLEY, Grenoble Cedex 9 (FR); Eva MONROY, Grenoble Cedex 9 (FR)

(73) Assignees: Commissariat à l'Energie Atomique et aux Energies Alternatives, Paris (FR); Centre National de la Recherche Scientifique, Paris (FR)

(21) Appl. No.: 18/146,748

(22) Filed: Dec. 27, 2022

(30) Foreign Application Priority Data
Dec. 27, 2021 (FR) 21 14563

(51) Int. Cl. G01N 33/543; G01N 33/58; B82Y 15/00

(52) U.S. Cl. CPC G01N 33/54386; G01N 33/582; B82Y 15/00

(57) ABSTRACT

The invention is a nanowire-comprising device for detecting at least one target biomolecule. The nanowires are placed between a substrate and a multilayer structure. The multilayer structure lies between the nanowires and a sample liable to contain a target biomolecule. The multilayer structure comprises a functionalization surface making contact with the sample. When light is emitted from the functionalization surface, fluorescence light for example, at least one nanowire allows the light to be detected. The fluorescence light may indicate the presence or absence of a target biomolecule on the functionalization surface.

### TECHNICAL FIELD

The technical field of the invention is detection of biomolecules through optical transduction performed by nanowires.

### PRIOR ART

Devices allowing biomolecules to be detected may be based on optical detection. The biomolecules to be detected are labelled beforehand using a fluorophore or quantum-dot luminescent label. Detection is achieved via a CCD or CMOS matrix-array photodetector or a matrix array of avalanche photodetectors (APDs). However, conventional matrix-array photodetectors employ a detection array the spatial pitch of which is generally larger than 1 μm. By spatial pitch, what is meant is the distance between two adjacent pixels. Such a spatial pitch is considered to be too large to obtain a device allowing a high number of detections to be addressed in parallel. These devices also have a limited sensitivity, which seems to be insufficient to detect sequences after a low number of amplifications.

Certain biosensors are based on detection of charge carried by the target biomolecules to be detected. It is for example a question of field-effect transistors. This type of device allows rapid detection, with a good sensitivity. In addition, CMOS technology (CMOS standing for complementary metal-oxide-semiconductor) allows devices comprising a high number of sensors to be manufactured. It is thus possible to carry out a high number of analyses in parallel. However, this type of sensor may be sensitive to environmental parameters affecting the sample, for example pH or temperature or interactions with non-targeted biomolecules or ions inducing detection errors. Use of this type of biosensor in DNA-sequencing applications (DNA standing for deoxyribonucleic acid) requires a certain measurement redundancy, so as to increase the robustness of the measurements. DNA sequencers based on nanopores have the same drawbacks.

Photonic sensors based on nanowires have been developed for the purposes of detecting fragments of DNA. This is for example the case of the publication Sing. et al “Silicon Nanowire Optical Rectangular Waveguide Biosensor for DNA Hybridization”, IEEE Photonics Technology Letters, 2018, 30(12) 1123-1126. In this publication, DNA hybridization is detected by detecting a variation in refractive index during the hybridization.

The publication Irrera. “New Generation of Ultrasensitive Label-Free Optical Si Nanowire-Based Biosensors”, ACS Photonics 2018, 5, 471-479 describes a nanowire-based biosensor used to detect CRP (C-reactive protein) in human serum.

The inventors have designed a compact and sensitive device for analysing a sample, allowing advantage to be taken of the optical detection capacities of nanowires. The device may be configured to allow various biomolecules to be analysed simultaneously or sequentially.

### SUMMARY OF THE INVENTION

A first subject of the invention is a device for detecting at least one biomolecule of interest in a sample, the device comprising:
a substrate, comprising at least a first electrode;
a multilayer structure, comprising at least a second electrode;
nanowires, extending between the first electrode and the second electrode, parallel to a transverse axis;
an encapsulation layer extending around the nanowires, between the substrate and the multilayer structure, the encapsulation layer being formed from an insulating material;
the multilayer structure comprising:
   a conductive layer, forming each second electrode;
   an electrically insulating interface layer covering each second electrode, each second electrode being interposed between the interface layer and a nanowire, the interface layer being bounded by a functionalization surface, the interface layer being configured to be placed between the sample and the second electrode, such that the functionalization surface forms an interface between the device and the sample;
the multilayer structure being such that
   the functionalization surface is configured to selectively capture the biomolecule of interest;
   the second electrode and the interface layer are transparent in a detection spectral band;
      wherein:
each nanowire comprises a homojunction, or heterojunction, or Schottky junction between the first electrode and the second electrode;
the first electrode and the second electrode are configured to be connected to a detection circuit;
such that each nanowire forms a nanophotodetector of light emitted from the functionalization surface, the light detected by each nanowire inducing a detection electrical signal in the detection circuit.

Thus, the interface layer allows the nanowires to be isolated from the sample.

Each nanowire may comprise:
a p-n homojunction;
a heterojunction;
a p-metal or n-metal Schottky junction.

At least one nanowire may comprise a first portion and a second portion, which are separated by the junction, the first portion being formed from an n-doped semiconductor, the second portion being formed from a p-doped semiconductor, the junction forming a homojunction or a heterojunction.

At least one nanowire may comprise a first portion and a second portion, which are separated by the junction, the first portion being formed from a semiconductor, the second portion, adjacent an electrode, being formed from a metal, the junction forming a Schottky junction.

According to one embodiment, the first portion and the second portion lie, along the transverse axis, on either side of the junction.

According to one embodiment, the second portion encircles the first portion, around the transverse axis.

According to one embodiment, at least one nanowire comprises a quantum well, or a quantum dot at the junction.

According to one embodiment, the functionalization surface is segmented into various elementary surfaces, each elementary surface forming a site of capture of the biomolecule of interest.

According to one embodiment,
the interface layer comprises two sublayers, stacked on each other, forming a lower sublayer and an upper sublayer, the lower sublayer being interposed between the conductive layer and the upper sublayer;
the upper sublayer comprises wells, opening into the lower sublayer, each well being placed facing one nanowire, each well forming one portion of the functionalization surface;
the functionalization surface is segmented level with each well, so that each well forms a site of capture of the biomolecule of interest.

Between the wells, the upper sublayer may not be functionalized, or comprise a coating preventing deposition of a biomolecule of interest.

According to one embodiment, the device comprises a plurality of nanowires, extending between the same first electrode and the same second electrode, the nanowires forming a bunch of nanowires. The device may comprise a plurality of bunches of nanowires, which are separated from one another, such that any nanowire of a bunch is closer to another nanowire of said bunch than to another nanowire of another bunch, the nanowires of a given bunch extending between the same first electrode and the same second electrode.

According to one embodiment, referred to as the multiplexing embodiment, the device comprises a plurality of nanowires, the device being such that:
a plurality of first electrodes are formed on the substrate, and a plurality of second electrodes are formed on the multilayer structure, each nanowire extending between a first electrode and a second electrode;
each first electrode is connected to a first addressing unit, configured to select at least one first electrode;
each second electrode is connected to a second addressing unit, configured to select at least one second electrode;
such that the detection circuit detects a detection signal induced by each nanowire extending between the selected first electrode and the selected second electrode.

The first addressing unit may be configured to successively select a plurality of first electrodes. The second addressing unit may be configured to successively select a plurality of second electrodes.

According to one possibility,
the substrate and the multilayer structure extend parallel to a longitudinal axis and to a lateral axis, the lateral axis and the longitudinal axis being secant to each other, in a plane secant to the transverse axis;
each first electrode is placed parallel to the longitudinal axis;
each second electrode is placed parallel to the lateral axis.

Advantageously,
the functionalization surface is functionalized by a biological detection probe, the biological detection probe being configured to selectively capture a biomolecule of interest;
such that capture of the biomolecule of interest leads to a variation in the fluorescence light detected by at least one nanowire when the functionalization surface is illuminated with excitation light to excite a fluorescent label grafted to the biomolecule of interest or to the biological detection probe.

The biological detection probe may be a molecular beacon.

According to one possibility,
the functionalization surface is segmented into a plurality of segments;
each segment of the functionalization surface lies facing at least one nanowire;
various segments of the functionalization surface are functionalized by biological detection probes configured to selectively capture various biomolecules of interest, respectively.

Another subject of the invention is a method for detecting at least one biomolecule of interest using a device according to the first subject of the invention, the functionalization surface being functionalized beforehand with a biological detection probe configured to selectively capture the biomolecule of interest, the device comprising:
a) placing a sample, liable to contain the biomolecule of interest, in contact with the functionalization surface;
b) connecting the detection circuit to the terminals of a first electrode and of a second electrode;
c) exposing the functionalization surface to excitation light, in an excitation spectral band of a fluorescent label, the fluorescent label being grafted to the biological detection probe or to the biomolecule of interest, the fluorescent label being capable of emitting fluorescent light, in the detection spectral band, when it is illuminated by the excitation light;
d) depending on the detection signal, detecting a variation in fluorescence light detected by at least one nanowire, the variation indicating capture of the biomolecule of interest by a biological detection probe.

According to one embodiment,
the device is a device according to the multiplexing embodiment described with reference to the first subject of the invention;
steps b) to d) are reiterated, modifying, between two successive iterations, the selected first electrode or the selected second electrode.

The invention will be better understood on reading the examples of embodiment that are presented, in the rest of the description, with reference to the figures listed below.

### FIGURES

FIGS. 1A to 1D show the main components of a device according to the invention.

FIGS. 2A to 2E schematically show steps of nanowire fabrication in a so-called bottom-up process.

FIGS. 3A to 3C show various possible arrangements of the nanowires.

FIGS. 4A and 4B schematically show steps of nanowire fabrication in a so-called top-down process.

FIG. 5 shows one example of a device according to the invention, in which example the electrodes are distributed in a matrix-array arrangement.

FIGS. 6A to 6E schematically show various modes of coupling between a biological detection probe and a target biomolecule, such that the capture of the target biomolecule induces emission or arrest of fluorescence light.

FIGS. 7A and 7B schematically show a molecular beacon before and after capture of the target biomolecule.

FIGS. 8A and 8B show two different nanowire structures.

### DESCRIPTION OF PARTICULAR EMBODIMENTS

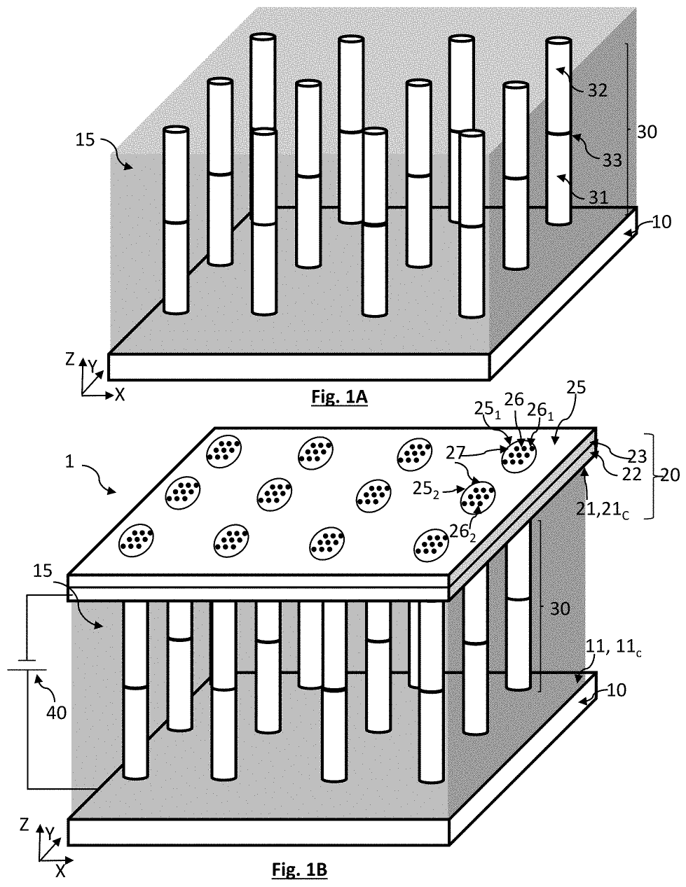

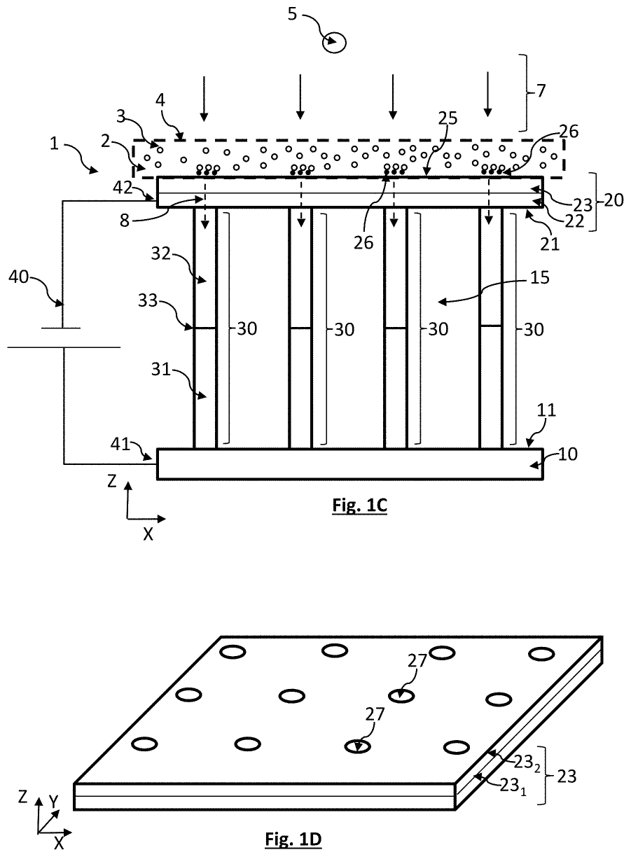

FIGS. 1A to 10 show a first example of an analysing device 1 allowing the invention to be implemented. The analysing device 1 is configured to be placed in contact with a sample 2, for example comprising a liquid medium liable to contain a biomolecule of interest 3. The biomolecule of interest 3 may be chosen from: a protein, an enzyme for example; a protein fragment; an antibody; an antigen; a single strand of nucleotides; and a hormone.

The device comprises a substrate 10, forming or comprising at least a first electrode 11_{c}. In the example shown in FIG. 1A, the substrate is a crystalline silicon substrate, for example an Si substrate of (111) crystal orientation. The substrate 10 is bounded by a surface, called the first surface 11, comprising the first electrode 11_{c}. In the example of FIG. 1A, the first surface 11 is formed from Si comprising conductive regions. According to another possibility, the substrate 10 is subjected to a deposition of a conductive layer, of graphene for example, forming all or some of the first surface 11.

Nanowires 30 are formed on the substrate 10, and more precisely from the first surface 11. The first surface 11 lies in a plane P_{XY}. The plane P_{XY} is defined by a longitudinal axis X and a lateral axis Y. The axes X and Y are secant, and preferably perpendicular to each other. The nanowires extend parallel to a transverse axis Z secant to the plane. In the embodiments described below, the transverse axis is perpendicular to the plane P_{XY}. The first surface 11 is conductive at least at the intersection with each nanowire 30. The entirety of the first surface 11 may be conductive.

In other configurations, the nanowires may be inclined and not perpendicular to the plane P_{XY}. For example, if the crystal orientation of the material forming the substrate 10 is (001), the nanowires may grow in a (111) direction, and therefore slant with respect to the plane P_{XY}.

The nanowires 30 preferably have a diameter comprised between 1 nm and 500 nm and a height, along the transverse axis Z, comprised between 300 and 1000 nm, or even 10000 nm.

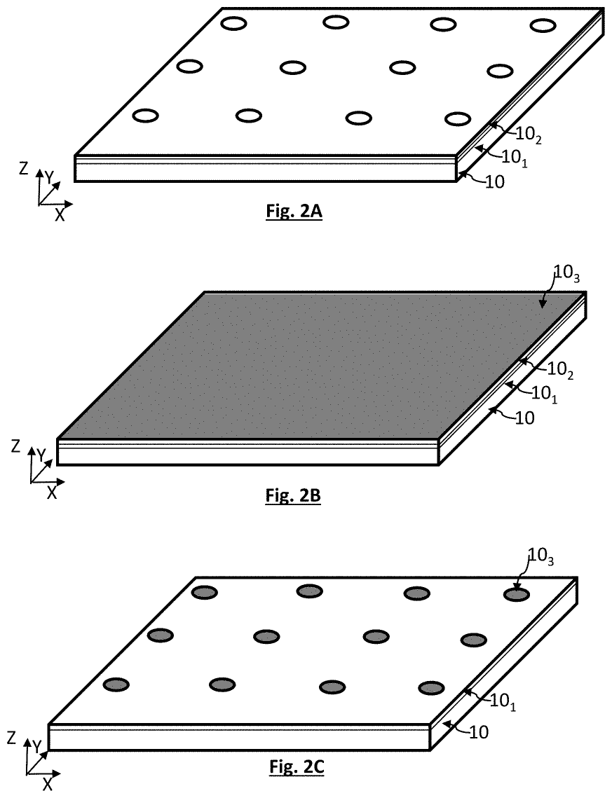

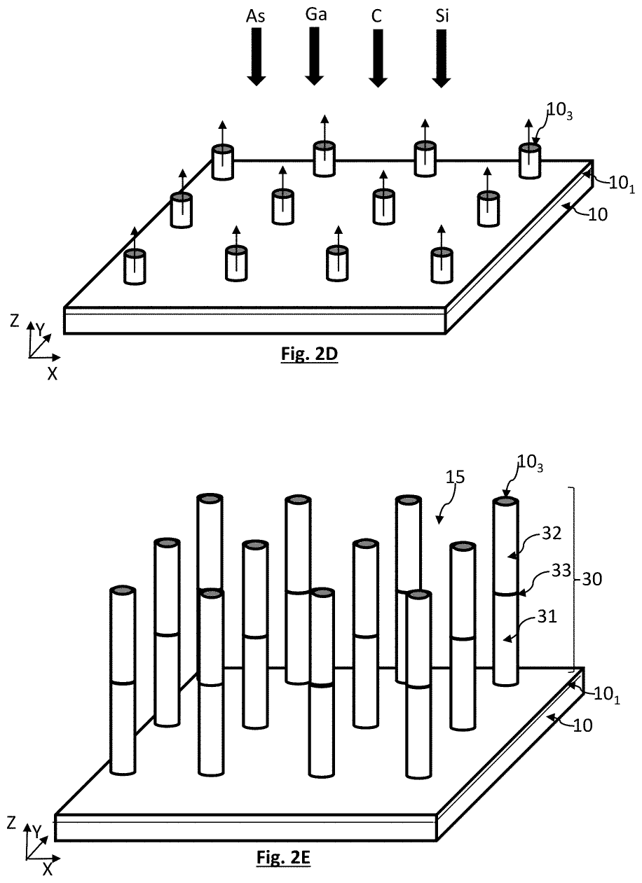

The nanowires 30 may be synthesized directly on the substrate 10, as described with reference to FIGS. 2A to 2E. The nanowires may be formed on another substrate, then transferred to the substrate 10. The transfer may be carried out as described in the publication Valente J. et al “Light-Emitting GaAs Nanowires on a Flexible Substrate”, Nano Lett 2018 18 7 4206-4213.

The nanowires 30 extend, from the first surface 11, to a second surface 21 bounding a multilayer structure 20. Just like the first surface 11, the second surface 21 is conductive at least at the intersection with each nanowire 30. In the example shown in FIG. 1B, the second surface 21 is formed from a layer 22 of a conductor that is transparent in a detection spectral band described below. It may for example be a question of ITO (indium tin oxide).

Each nanowire is formed from one or more semiconductors, and optionally from a metal. Each nanowire comprises a junction 33. In the example shown, the junction 33 is a homojunction: each nanowire comprises a first portion 31, adjacent the first surface 11, and a second portion 32, adjacent the second surface 21. The first and second portions are formed from the same semiconductor, but doped differently: thus, the first portion 31 and the second portion 32 are formed from the same semiconductor, but doped n-type and p-type or p-type and n-type, respectively. In the example shown, the first portion 31 is formed from p-doped gallium arsenide (GaAs) and the second portion is formed from n-doped GaAs. The interface between the two portions forms the (p-n) junction 33.

Alternatively, the junction 33 may be formed in a metal placed on or in contact with the surface 21 so as to produce a Schottky semiconductor-metal junction.

Each nanowire comprises at least one semiconductor chosen from III-V semiconductors, i.e. semiconductors made of elements from columns III and V, GaAs for example. It may preferably be a question of a semiconductor made of an element from column III and of arsenic, InAs (indium arsenide) for example. In the example shown, the nanowires 30 are formed from GaAs. Other semiconductors may be envisaged, for example, and non-limitingly, Si, InGaAs, AlGaAs, InGaP, InGaN, GaN, ZnSe, ZnS, ZnO, and ZnCdO.

Between the first surface 11 and the second surface 21, the nanowires 15 are embedded in an encapsulation layer formed from an insulating material. The encapsulation layer 15 may be formed from a material such as PMMA (polymethyl methacrylate), BCB (benzocyclobutene), or SiO₂. The encapsulation layer 15 may be deposited by spin-coating.

The encapsulation layer 15 is preferably formed following growth of the nanowires, prior to deposition of a second conductor 22, which is bounded by the second surface 21 and intended to form the second electrodes 21_{c}. The second electrodes 21_{c} are formed, on the second surface 21, from the conductive layer 22. The conductive layer 22 may be structured so that on the second surface 21, a plurality of second electrodes 21_{c} are electrically isolated from one another. Thus, the conductive layer may comprise apertures or insulating materials bounding the electrodes 21_{c}.

Apart from the conductive layer 22, the multilayer structure 20 comprises an interface layer 23, adjacent the conductive layer 22. The interface layer 23 is transparent. The interface layer 23 may for example be formed from a polymer, PMMA (polymethyl methacrylate) for example. The interface layer 23 is insulating, in particular at the interface with the conductive layer 22. The interface layer 23 is preferably planar.

The interface layer 23 is intended to form an interface between the conductive layer 22, forming the electrodes 21_{c}, and the sample 2. Thus, the interface layer 23 extends between the conductive layer 22 and the sample 2 to be analysed. One important aspect of the device is that the sample does not make direct contact with the nanowires. It is isolated from the latter by the interface layer 23.

The interface layer 23 is for example a thin layer formed from SiO₂ or from PMMA. The surface of the interface layer 23 intended to make contact with the sample is a surface, referred to as the functionalization surface 25, that is functionalized by biological detection probes 26. It is important for the interface layer 23 to be formed from a material having the lowest possible autofluorescence in the detection spectral band.

The functionalization surface 25, which forms an interface between the multilayer structure 20 and the sample 2 to be analysed, is intended to be functionalized by biological detection probes 26. By biological detection probe, what is meant is a species configured to capture a biomolecule of interest selectively. The biological detection probe 26 may be formed from nucleic acids, or from proteins, or from antibodies, or from antigens, or from enzymes. Selective capture is achieved through coupling of the biomolecule of interest to the detection probe, via specific interactions: hybridization (DNA, RNA), folding (aptamers), multiple low-energy chemical interactions (antibodies, enzymes), adsorption (proteins).

The interface layer 23 may be nanostructured, such that nanowells 27 are formed at the interface between the multilayer structure 20 and the sample to be analysed. The diameter or the largest diagonal of the nanowells may be comprised between 70 nm and 700 nm. The nanowells 27 may for example be arranged in a matrix array, or, more generally, in a predetermined pattern. The functionalization surface is then functionalized in each nanowell 27, the spaces between each well not being functionalized. The nanowells may be formed by thinning the interface layer 23 locally.

According to one possibility, shown in FIG. 1D, the interface layer 23 comprises two superposed sublayers 23₁ and 23₂. The interface layer comprises a lower sublayer 23₁ interposed between the conductive layer 22 and an upper sublayer 23₂. The nanowells are formed by thinning the upper sublayer 23₂ locally, so that the nanowells open into the lower sublayer 23₁. The upper sublayer 23₂ may be formed from a non-functionalizable material, an anti-biofouling material for example. Such structuring allows the functionalization surface 25 to be functionalized only in the nanowells 27, on the lower sublayer 23₁. Each nanowell 27 thus forms a site of capture of the biological species of interest 3.

More generally, the functionalization surface 25 may be functionalized in a predetermined functionalization pattern. Outside of the functionalization pattern, the functionalization surface is not functionalized with respect to the biological species that it is desired to capture.

Functionalization may be achieved via a treatment of the functionalization surface 25, a plasma/oxygen surface treatment for example. When the interface layer 23 is formed from PMMA, a plasma/oxygen treatment allows carboxyl functions to be formed. The biological detection probes may be grafted to the functionalization surface via covalent bonding. To this end, the biological probes comprise a function, for example an amine function, so as to form a component bond with the functions of the functionalization surface 25.

When the functionalization surface 25 is functionalized in a spatial pattern, the portions of the functionalization surface outside of the spatial pattern may be coated with an anti-biofouling coating so as to prevent non-specific adsorption of the biomolecules of interest (or targets). One example of an anti-biofouling material is polyethylene glycol.

The biological detection probes may be deposited through chemical coupling to the functionalization surface 25.

According to one possibility, various portions 25₁, 25₂ of the functionalization surface 25 are functionalized with various biological detection probes 26₁, 26₂. Each portion of the functionalization surface 25 is thus functionalized so as to selectively capture a different biomolecule of interest from another portion of the functionalization surface. Preferably, a given portion of the surface is functionalized with the same biological detection probe intended to capture a given biomolecule of interest. For example, when the biological detection probes are formed from single-strand DNA fragments, a given portion of the functionalization surface is functionalized with the same DNA fragment. Two different portions are functionalized with different DNA fragments. Prior to the functionalization, the DNA fragments forming the biological detection probes are prepared using prior-art methods, for example preparation of a library of short DNA fragments (<150 bp−bp=base pairs) from a long strand extracted from biological cells. Thus, the device may simultaneously or sequentially address various biological species of interest.

When various portions of the functionalization surface are functionalized with various detection probes, the device allows a so-called multiplex analysis to be performed: the analysis may simultaneously or successively address various biomolecules of interest, for example various sequences of nucleotides.

As indicated above, each biological detection probe 26 is configured to capture one biomolecule of interest 3. By capture, what is meant is formation of a bond between the detection probe and the biomolecule of interest, leading to coupling of the biomolecule of interest to the detection probe. The bond between the detection probe and the biomolecule of interest may be a chemical bond (dative bond, hydrogen bond, disulfide bridge), or an antigen-antibody bond, or a physical bond (electrostatic interactions, hydrophobic interactions, or Van der Walls interactions) or a hybridization when the detection probe is a sequence of nucleotides and the biomolecule of interest is a complementary sequence of nucleotides.

The device 1 is based on optical detection of capture of a biomolecule of interest by a biological detection probe 26, inducing an electrical response from the device. The device comprises a detection circuit 40, a first terminal 41 of which is connected to a first electrode 11_{c}, on the substrate 10, and a second terminal 42 of which is connected to a second electrode 21_{c}, on the multilayer structure 20. The detection circuit 40 allows the potential difference, or an electric current, to be measured between the first electrode 11_{c} and the second electrode 21_{c}.

In FIG. 10, a cross-sectional view of the device has been shown. A liquid sample 2 is placed in a fluidic chamber 4, in contact with the interface layer 23. The sample is isolated from the nanowires 30 by the interface layer 23. Thus, the nanowires do not make contact with the biomolecules of interest.

When the device is in operation, a light source 5 illuminates the functionalization surface 25, in an excitation spectral band centred on a wavelength of excitation of fluorescence. In the example shown in FIG. 10, the light source generates excitation light 7, which propagates through the sample 2 to the functionalization surface 25. The biomolecule of interest 3 has been labelled beforehand with a fluorescent label 6. Under the effect of illumination at the excitation wavelength 7, the fluorescent label 6 generates fluorescence light 8 at a fluorescence wavelength, that is longer than the excitation wavelength. The fluorescent label 6 may be a fluorophore or a quantum dot.

Generally, capture of the biomolecule of interest leads to a variation in emission of fluorescence light 8, this variation in fluorescence leading to a variation in the electrical response of the device.

In the example shown in FIG. 10, capture of the biomolecule 3 by one of the detection probes 26 leads to concentration of fluorescent labels 6 on the functionalization surface 25, plumb with the nanowires. Under the effect of the excitation light 7, fluorescence photons are emitted, forming the fluorescence light 8. Because of the transparency of the interface layer 23, or of the material 22 from which the electrode 21_{c} is made, certain fluorescence photons propagate through a nanowire 30. When a fluorescence photon is absorbed at the junction 33, electron/hole pairs are formed. Under the effect of the potential difference between the first portion 31 and the second portion 32, electrons propagate through the n-doped segment, towards the higher potential, whereas the holes propagate in the opposite direction through the p-doped segment. An increase in the electrical current flowing through the detection circuit 40 results.

According to another possibility, capture of the biomolecule induces a decrease in or arrest of emission of the fluorescence photons, for example via a quenching effect. In the absence of capture, the fluorescence photons are emitted, this leading to a flow of an electrical current through the detection circuit 40. Following capture, emission of the fluorescence photons is decreased, or stopped, this leading to a variation in the electrical current.

It will be understood that the semiconductor chosen to form the nanowire 30 depends on the absorption spectral band of said material, said band having not only to contain the fluorescence wavelength, but also, preferably, not contain the excitation wavelength of the fluorescent label. The nanowire thus acts as nanophotodetector of the fluorescence light, while not being sensitive to the excitation wavelength. Each nanowire thus forms a filter with respect to the excitation wavelength. When the semiconductor is GaAs, the fluorescent label may be Cy3 (cyanine 3): excitation wavelength 540 nm and emission wavelength comprised between 555 and 600 nm.

Alternatively, when the nanowire is sensitive to the excitation wavelength, detection of the fluorescence light may be temporally separated from detection of the excitation wavelength.

The diameter of the nanowires is controlled so as to create sensitivity in a narrow spectral band. By narrow spectral band, what is meant is a bandwidth typically of a few tens of nm, and preferably narrower than 100 nm. The bandwidth corresponds to the full width at half maximum of the absorption peak in the absorption spectrum. The detection bandwidth is for example of the order of 50 nm. It is thus possible to adjust the detection spectral band, so that it contains the fluorescent wavelength of the fluorescence label and so that it does not contain the excitation wavelength of the fluorescent label. The ability of nanowires to form a wavelength-selective photodetector has been described in Mokkapati. S. et al “Optical design of nanowire absorbers for wavelength selective photodetectors”, Sci. Rep. 5, 15339.

Spectral detection sensitivity may be adjusted by incorporating quantum wells or quantum dots into the junction 33. This allows a junction 33 the composition of which is different from the rest of the nanowire to be obtained. The detected wavelength then corresponds to the wavelength of the bandgap defined by the quantum well or quantum dot.

Each nanowire thus forms a nanophotodetector. The invention is based on a combination of selective and spatialized capture of a target biomolecule and of detection of capture by nanophotodetectors. By using various detection probes distributed over various spatial regions of the functionalization surface to selectively address various target biomolecules, the invention allows the presence of various biomolecules to be detected simultaneously or successively.

Various aspects of the design of the device according to the invention will now be described.

### Formation of the Nanowires

FIGS. 2A to 2E schematically show steps for forming nanowires from a silicon substrate 10. FIGS. 2A to 2E correspond to a bottom-up approach. The substrate 10 is formed from Si, of (111) orientation, and comprises an SiO₂ surface layer 10₁ of 10 nm-15 nm thickness intended to form a barrier layer. The SiO₂ layer is covered by a PMMA layer 10₂ of 45 nm thickness. The layers 10₁ and 10₂ are subjected to an etch, so as to form nanowells that are isolated from one another, in a predetermined pattern. See FIG. 2A. The nanowells open onto the Si substrate. A thin metal layer 10₃, for example of gold, of 5 to 10 nm thickness is deposited on the SiO₂ layer. The added metal, in the present case gold, plays the role of catalyst. See FIG. 2B. The excess of gold between the nanowells is removed by lift-off of the PMMA layer 10₂. See FIG. 2C. Thus, gold islands 10₃ that are isolated from one another are obtained in positions corresponding to the position of the nanowells formed beforehand.

After heating to a temperature above 450° C., the islands form droplets. The gold droplets play the role of catalyst. The nanowires are then formed by MBE (molecular beam epitaxy). This consists in sending one or more molecular jets towards the substrate in order to achieve epitaxial growth. See FIG. 2D. The vapour-phase molecular jets contain the chemical species from which the semiconductor nanowire will be made and dopant species. The molecular species strike and diffuse into the gold droplets. When said species reach saturation, nucleation of the nanowires occurs first at the interfaces between the droplets and substrate, then at the interfaces between the droplets and the nanowires in formation.

The process does not have to use gold as the catalyst. Other catalysts may be used, Ga for example. When the catalyst is Ga and Ga is also a constituent element of the semiconductor, the process is referred to as self-catalysed nanowire growth.

This process for example allows nanowires to be grown using As and Ga, and C and Si is p-type and n-type dopant, respectively, the growth temperature being 610° C. The process continues until the nanowires reach a predetermined height. See FIG. 2E.

Following the steps shown in FIG. 2E, the encapsulation layer 15 is formed between the nanowires, for example by spin-coating. The encapsulation layer allows the nanowires to be electrically isolated from one another, and makes the assembly stronger. The end of the nanowires opposite the substrate may be polished and/or plasma etched so as to remove residues of the catalyst and to increase the uniformity of the height of the nanowires and of the encapsulation layer 15.

The conductive layer 22, then the interface layer 23, are then deposited in succession on the assembly formed by the nanowires and the encapsulation layer 15.

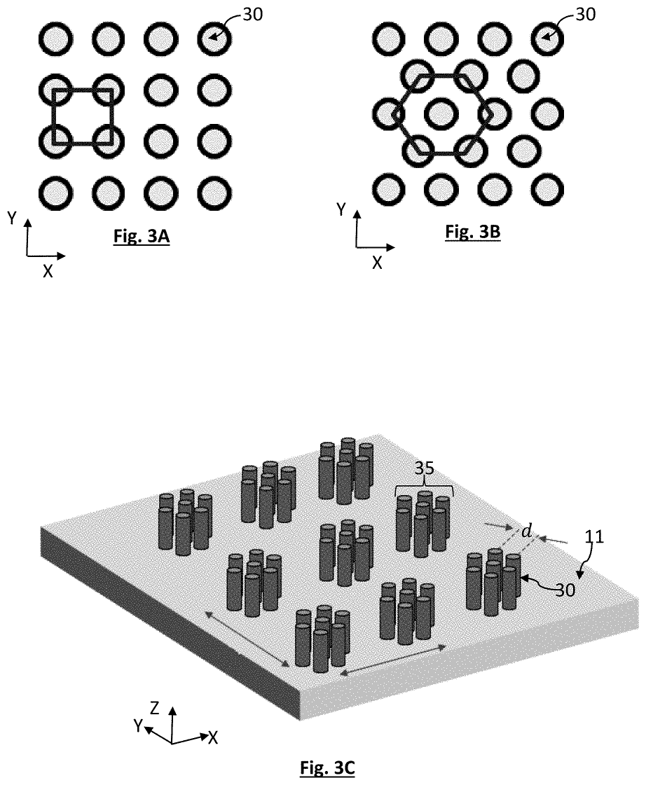

In the embodiment illustrated in FIGS. 2A to 2E, forming the nanowires on the substrate 10 allows the position of the nanowires to be controlled. Thus, the nanowires may be arranged regularly, as shown in FIGS. 3A and 3B. FIGS. 3A and 3B show nanowires positioned on the substrate 10 in patterns of square and hexagonal unit cells, respectively. The pitch between two adjacent nanowires may be small, of the order of two times their diameter, or may be larger, for example a few to several hundred nm.

Another advantage of the bottom-up approach described with reference to FIGS. 2A to 2E is that it allows the crystal structure of the nanowires to be precisely controlled. The bottom-up approach allows controlled incorporation of quantum dots or quantum wells, and in particular allows their position along the Z-axis to be controlled.

FIG. 3C illustrates a configuration in which the nanowires are arranged to form bunches 35, or clusters. The nanowires of a given cluster are closely spaced, though the distance d between two adjacent nanowires of a given bunch is preferably larger than or equal to the diameter of the nanowires. The distance between two adjacent bunches may be equal to or larger than two times the distance d. Each bunch may have a regular arrangement, as shown in FIGS. 3A and 3B.

The arrangement of the nanowires into clusters 35 may be combined with structuring of the functionalization surface into nanowells 27, as described with reference to FIG. 1B. In this case, each nanowell 27 lies opposite nanowires belonging to a given cluster 35.

It is not essential to place the nanowires in a predefined arrangement on the substrate 10. According to one possibility, droplets of metal catalyst are distributed randomly over the substrate 10. Following addition, in vapour phase, of the molecules from which the nanowire will be made, for example Ga and As and dopants, C and Si for example, the nanowires develop from the positions initially occupied, on the substrate, by the droplets of catalyst.

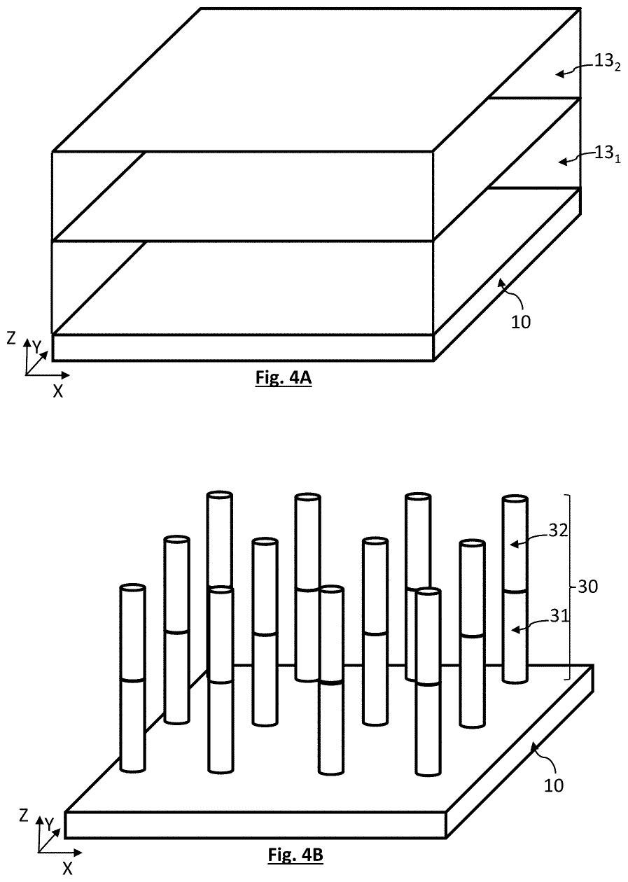

According to another possibility, the nanowires are obtained by etching, using a so-called top-down approach. With such an approach, a first layer 13₁ of a semiconductor doped a first type, n-doped for example, then a second layer 13₂ of a semiconductor doped another type, p-doped for example, are formed on the substrate 10 (see FIG. 4A). The nanowires are formed by etching the first layer 13₁ and the second layer 13₂. See FIG. 4B. An encapsulation layer 15 is then formed around the nanowires, as described above. The conductive layer 22 is then deposited on the free end of the nanowires 30, to form the electrodes 21c. The interface layer 23 is formed on the conductive layer 22. Such a mode of fabrication may also allow nanowires comprising a metal-semiconductor junction to be formed: the first layer 13₁ or the second layer 13₂ is formed from a metal, whereas the other layer is formed from a semiconductor.

The top-down approach allows nanowires the composition of which is uniform in terms of dimensions and of doping profile to be obtained. This is favourable to large-scale integration into devices.

### Electrical Addressing

As described with reference to FIGS. 1A to 10, each nanowire extends between a first end, on a first surface 11 of the substrate 10, and a second end, on a second surface 21 of the multilayer structure 20. The first surface 11 and the second surface 21 are conductive at least at each intersection with a nanowire. Thus, at each intersection with a nanowire, the first surface comprises a first electrode 11_{c} and the second surface comprises a second electrode 21_{c}. In the example shown in FIGS. 1A to 10, the first surface 11 and the second surface 21 are formed from a conductor. They are conductive over their entire extent.

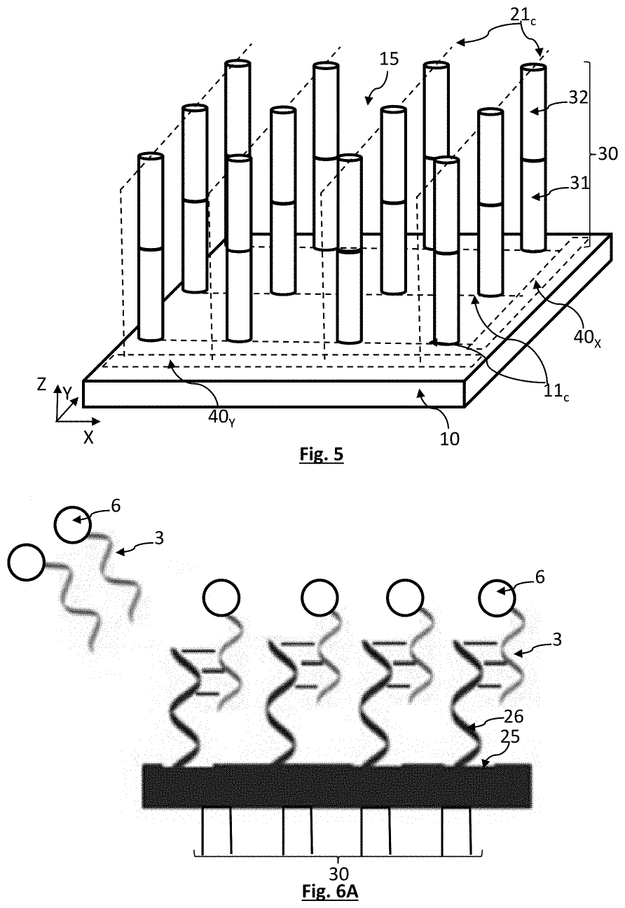

The first and second surfaces may be structured, and comprise various electrodes 11_{c}, 21_{c} that are isolated from one another. In the FIG. 5, each electrode has been represented by a dashed line. On the first surface 11, each first electrode 11_{c} describes a row, parallel to the longitudinal axis X. On the second surface 21, each second electrode 21_{c} describes a column, parallel to the lateral axis Y. Each nanowire is functional when the electrodes 11_{c}, 21_{c} between which it extends are biased. Structuring the electrodes into rows/columns allows the nanowires that will be functional to be selected, the latter extending between two biased electrodes: in this arrangement, the nanowires connected to a biased row and to a biased column are functional. By functional nanowire, what is meant is a nanowire biased to detect fluorescence photons.

A plurality of rows and/or a plurality of columns may be biased simultaneously or successively. The detection circuit 40 comprises:
an addressing unit 40_{X}, intended to bias all or some of the first electrodes 11_{c}, parallel to the axis X,
and an addressing unit 40_{Y}, intended to bias all or some of the second electrodes 21_{c}, parallel to the axis Y.

When the nanowires are distributed into clusters, as described with reference to FIG. 3C, the nanowires of a given cluster are preferably connected to the same electrode, both on the substrate 10 and on the multilayer structure 20. The nanowires of a given cluster are thus functional simultaneously. Nanowires not connected to the detection circuit are not functional.

### Functionalization—Fluorescence

The functionalization surface 25 is functionalized by grafting biological detection probes 26 to the functionalization surface 25. The biological detection probes 26 are intended to selectively capture a biomolecule of interest 3. In one multiplexing embodiment, various biological detection probes 26, respectively addressing various biomolecules of interest, are located separately on the functionalization surface 25. With reference to FIG. 3C, the portion of the functionalization surface located facing a given cluster 35 may be functionalized with the same biological detection probe 26, so as to address a given biomolecule of interest 3. Advantage is then taken of potential detection, by a plurality of adjacent nanowires, of the same biomolecule of interest.

Two different portions 25₁, 25₂ of the functionalization surface 25, respectively placed facing two different clusters, may be functionalized with two different biological detection probes, so as to address two different biomolecules of interest. Such a configuration is particularly suitable for detecting various target nucleotide sequences. The biological detection probe then comprises a nucleotide sequence complementary to the target nucleotide sequence. Because of the high sensitivity of each nanowire, the number of nanowires in a given bunch may be relatively low. Thus, the area, in the plane P_{XY}, of each bunch is small, each bunch selectively addressing a different target nucleotide sequence of another bunch. It is thus possible to provide a high number of bunches, respectively addressing various target nucleotide sequences, in the same compact device.

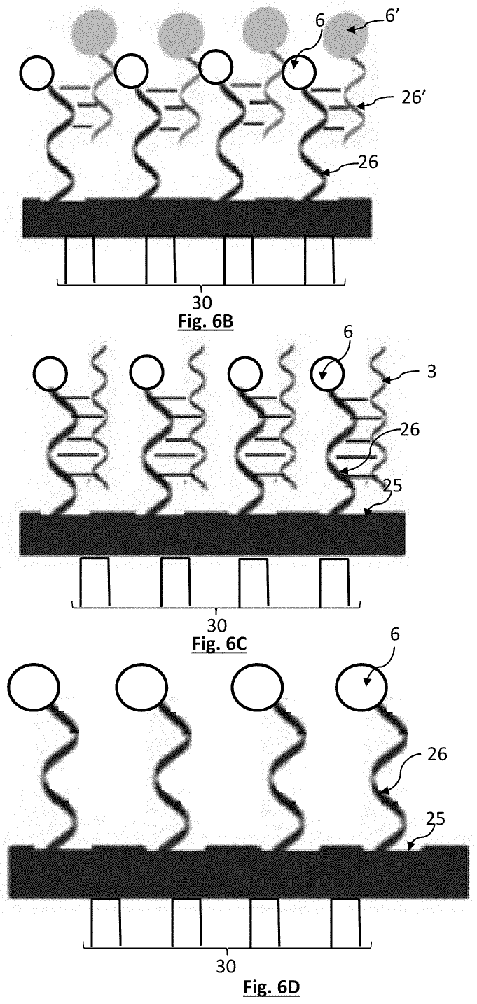

According to one possibility, shown in FIG. 6A, each biomolecule of interest 3 is labelled with a fluorescent label 6, as described above. In this case, the accumulation of captured biomolecules of interest 3 in the vicinity of the functionalization surface 25 engenders fluorescence light that is detectable by the closest nanowires 30. Capture of the biomolecules of interest leads to an increase in the fluorescence light, which is perceptible by the detection circuit 40. In FIG. 6A, the biomolecule of interest 3 is a DNA sequence complementary to a DNA strand forming the biological detection probe 26. The biomolecule of interest is captured via hybridization of the complementary strand with the strand forming the biological detection probe 26.

According to another possibility, shown in FIGS. 6B and 6C, the biological detection probe 26 is labelled using a fluorescent label 6. It is also bonded to a complementary structure 26′ bearing a fluorescence quencher 6′. In the presence of a quencher (see FIG. 6B), energy is transferred between the fluorophore and the quencher. The biological detection probe 26 has a higher affinity to the biomolecule of interest than to the complementary structure 26′. In the absence of the biomolecule of interest 3, or in the presence of an insufficient amount of the biomolecule of interest, from/in the sample 2, the action of the quencher results in a negligible emission of fluorescence light by the biological detection probe 26. In the presence of a biomolecule of interest 3 adsorbed on the detection probe 26 (see FIG. 6C), said biomolecule tends to fasten to the biological detection probe 26, replacing the complementary structure 26′. Disappearance of the quencher 6′ leads to an emission of fluorescence light by the fluorescent label 6, indicating capture of the biomolecule of interest 3.

According to a possibility, shown in FIGS. 6D and 6E, each biological detection probe 26 is bonded to a fluorescent label 6 and the biomolecule of interest 3 is bonded to a quencher. In the absence of a biomolecule of interest 3 coupled to the detection probe 26, fluorescence light 8 is emitted. See FIG. 6D. In the presence of a biomolecule of interest 3 adsorbed on the detection probe 26, the quencher blocks the emission of the fluorescence light. Thus, capture of a biomolecule of interest results in a decrease in the fluorescence light. See FIG. 6E.

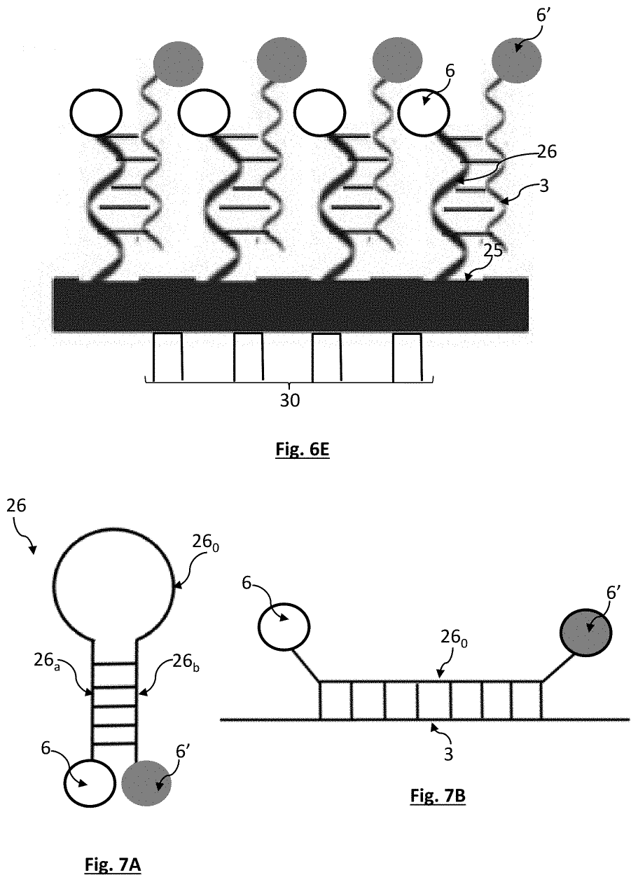

FIGS. 7A and 7B schematically show a preferred structure of a biological detection probe 26. Such a structure is usually designated a molecular beacon. This type of probe is intended to capture a single-strand nucleotide sequence. In FIG. 7A, the detection probe 26 comprises two complementary arms 26ₐ, 26_{b} extending parallel to each other, from a loop 26₀. A fluorescent label is grafted by covalent bonding at the end of one arm 26ₐ whereas a quencher is grafted by covalent bonding at the end of the complementary arm 26_{b}. The loop 26₀ consists of a sequence that is complementary to the biomolecule of interest 3. In the absence of a biomolecule of interest 3, the detection probe remains closed, its shape resembling that of a hairpin. The presence of the quencher 6′ prevents or limits the emission of fluorescence light by the fluorescent label 6. Adsorption of a biomolecule of interest 3 on the loop 26₀ causes the molecular beacon to open, as shown in FIG. 7B. The increased distance between the fluorescent label 6 and the quencher 6′ prevents energy transfer, leading to arrest of the fluorescent emission. Thus, the fluorescence light emitted by the fluorescent label 6 is emitted and may be detected by a nanowire. With this type of detection probe, capture of a biomolecule of interest results in an increase in fluorescence light.

For a molecular beacon such as schematically shown in FIGS. 7A and 7B to work, the affinity of the loop 26₀ to the biomolecule of interest must be high enough for the two complementary arms 26ₐ, 26_{b} to be able to be decoupled.

This type of molecular beacon may be grafted to the functionalization surface 25 through formation of carboxyl bonds with the detection surface. When the contact layer is formed from PMMA, carboxyl functions may be formed on its surface via an oxygen-plasma treatment. The beacons may comprise an amine function. The detection surface is functionalized by forming covalent bonds with the carboxyl function on the functionalization surface 25.

### Structure of the Nanowires

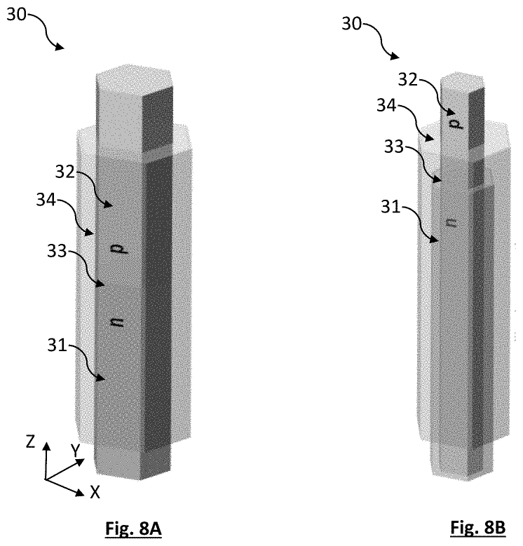

FIGS. 8A and 8B show other nanowire structures able to be employed in a device according to the invention. FIG. 8A shows a nanowire similar to the nanowires described above. The junction 33 is placed axially and lies between two differently doped portions 31, 32 that are spaced apart from each other along the transverse axis Z. In the example of FIG. 8A, a passivating cladding 34 encircles the nanowire.

In the example of FIG. 8B, the junction 33 extends radially between two differently doped regions. Thus, the junction 33 extends around the transverse axis Z, parallel to the latter. The first portion 31 and the second portion 32 are separate radially, the boundary between the two portions corresponding to a radius of separation. The first portion lies between the axis of the nanowire and the junction 33, whereas the second portion lies around the junction 33.

An axial structure is considered to be advantageous because it promotes incorporation of quantum wells or quantum dots inside the nanowires with a view to adjusting the absorption spectrum.

A radial structure allows a junction 33 to be provided that extends a substantial height along the axis Z, this allowing detection sensitivity to be increased. Optionally, the radial structure shown in FIG. 8B comprises an annular cladding 34 such as described with reference to FIG. 8A.

The nanowire structures described with reference to FIG. 8A may be produced using a top-down approach, for example one employing etching, as described with reference to FIGS. 4A and 4B, by adapting the make-up of the layers deposited on the substrate 10. The nanowire structures described with reference to FIG. 8B may be produced by nanowire growth.

### Applications

The device described above may be used to detect hybridization of two complementary DNA fragments, one forming the detection probe, the other forming the target biomolecule. Applications may concern PCR (polymerase chain reaction). It is believed that the device will allow more rapid detection of an amplification: specifically, the sensitivity of the device makes it possible to distinguish between one copy and two copies of the same fluorescent biomolecule, i.e. the very first sequence amplified by duplication may be detected, in contrast to prior-art devices, which require at least 5 amplification cycles (2⁵ copies). The device may be used in sequencing, high-throughput DNA sequencing for example. Advantage is taken of the sensitivity of the nanowires, which allows the required number of copies of an examined sequence of nucleotides to be decreased. In addition, as described above, the device allows a plurality of bunches of nanowires respectively addressing various sequences of nucleotides to be provided. This allows a plurality of in-parallel detections. Each bunch of nanowires is then associated with one nanowell 27 formed in the interface layer 23. Each nanowell is seeded with a single DNA strand obtained from a library obtained beforehand using known techniques. Each strand is simultaneously and competitively amplified (for example via bridge amplification) in order to colonize the entire surface of the nanowell with monoclonal strands, thus forming bunches of monoclonal DNA strands aligned with the bunches of nanowires. The DNA strands are then hybridized base by base with complementary bases using a so-called one-channel protocol comprising two cycles:
in the first cycle, for example, the bases A (adenine) and T (thymine) of the strand to be sequenced are hybridized with a complementary base, to which complementary base is grafted a fluorophore that emits at the wavelength detected by the nanowires. Fluorescence corresponding to the bases A and T is then recorded.
the second cycle makes it possible to dissever the fluorophore that allowed the bases A to be identified, and to preserve it on the bases T, and to graft a fluorophore to the bases C (cytosine). Fluorescence corresponding this time to the bases T and C is recorded, again.
through combination of the two recordings, the hybridized base is deduced for each monoclonal cluster: base A if a fluorescent signal was recorded in the first cycle but not in the second; base T if a fluorescent signal was recorded in both cycles; base C if a fluorescent signal was recorded in the second cycle but not in the first; base G (guanine) if no fluorescence was detected in both the first and second cycles.

This sequence is repeated as many times as there are bases to be sequenced.

When the device is used to detect DNA hybridization, the detection probes will preferably be labelled, rather than the targets. In this regard, use of molecular beacons, not requiring the targets to be labelled, is preferred.

The device may also be applied to detection of an antibody-antigen complex.

The device benefits from a rapid response time, typically of the order of one ns. In addition, use of various biological probes that respectively address various target biomolecules, and that are isolated from one another on the detection surface, allows various target biomolecules to be addressed simultaneously. It is possible to obtain, for each sample, a response that may be said to be digital: presence and absence of the various target biomolecules is detected simultaneously, the response being 1 in case of presence and 0 in case of absence of a detectable amount.

It may be seen that the device does not require bulky optical components to be employed. In addition, the response of the device is stable, and insensitive to environmental variations: pH of the sample, temperature, presence of molecules or ions different from the biomolecule of interest. This is due to the fact that the nanowires do not make contact with the sample, but are physically and electrically isolated from the latter by the interface layer 23.

Lastly, the device allows measurements to be taken without systematic calibration. For example, in ELISAs (ELISA standing for enzyme-linked immunosorbent assay), it is typically necessary to calibrate existing systems on each measurement of a dilution range, in order to overcome the overall instability of the system (optical components, etc.). Our system, because of its stability, needs be calibrated only once for all the measurements taken in a time frame guaranteeing the stability of the reagents: the latter become the only limiting element requiring periodic recalibration. Lastly, since the device is based on nanophotodetectors, it allows a compact analysis platform to be obtained. The device may be obtained using a wafer-level fabrication process, this allowing the cost to be decreased.

1. A device for detecting at least one biomolecule of interest in a sample, the device comprising:
a substrate, comprising at least a first electrode;
a multilayer structure, comprising at least a second electrode;
nanowires, extending between the first electrode and the second electrode, parallel to a transverse axis;
an encapsulation layer extending around the nanowires, between the substrate and the multilayer structure, the encapsulation layer being formed using an insulating material;
the multilayer structure comprising:
a conductive layer, forming each second electrode;
an electrically insulating interface layer covering each second electrode, each second electrode being interposed between the interface layer and a nanowire, the interface layer being bounded by a functionalization surface, the interface layer being configured to be placed between the sample and the second electrode, such that the functionalization surface forms an interface between the device and the sample;
the multilayer structure being such that
the functionalization surface is configured to selectively capture the biomolecule of interest;
the second electrode and the interface layer are transparent in a detection spectral band;
wherein:
each nanowire comprises a homojunction, or heterojunction, or Schottky junction between the first electrode and the second electrode;
the first electrode and the second electrode are configured to be connected to a detection circuit;
such that each nanowire forms a nanophotodetector of light emitted from the functionalization surface, the light detected by each nanowire inducing a detection electrical signal in the detection circuit.

2. The device according to claim 1, wherein each nanowire comprises at least one of:
a p-n homojunction,
a heterojunction;
a p-metal or n-metal Schottky junction.

3. The device according to claim 2, wherein at least one nanowire comprises a first portion and a second portion, which are separated by the junction, the first portion being formed from an n-doped semiconductor, the second portion being formed from a p-doped semiconductor, the junction forming the p-n homojunction or the heterojunction.

4. The device according to claim 2, wherein at least one nanowire comprises a first portion and a second portion, which are separated by the junction, the first portion being formed from a semiconductor, the second portion, adjacent an electrode, being formed from a metal, the junction forming the Schottky junction.

5. The device according to claim 2, wherein the first portion and the second portion lie, along the transverse axis, on either side of the junction.

6. The device according to claim 3, wherein the second portion encircles the first portion, around the transverse axis.

7. The device according to claim 1, wherein at least one nanowire comprises a quantum well, or a quantum dot at the junction.

8. The device according to claim 1, wherein the functionalization surface is segmented into various elementary surfaces, each elementary surface forming a site of capture of the biomolecule of interest.

9. The device according to claim 8, wherein:
the interface layer comprises two sublayers, stacked on each other, forming a lower sublayer and an upper sublayer, the lower sublayer being interposed between the conductive layer and the upper sublayer;
the upper sublayer comprises wells, opening into the lower sublayer, each well being placed facing one nanowire, each well forming one portion of the functionalization surface;
the functionalization surface is segmented level with each well, so that each well forms a site of capture of the biomolecule of interest.

10. The device according to claim 1, comprising a plurality of nanowires, extending between the same first electrode and the same second electrode, the nanowires forming a bunch of nanowires.

11. The device according to claim 10, comprising a plurality of bunches of nanowires, which are separated from one another, such that any nanowire of a bunch is closer to another nanowire of said bunch than to another nanowire of another bunch, the nanowires of a given bunch extending between the same first electrode and the same second electrode.

12. The device according to claim 1, comprising a plurality of nanowires, wherein:
a plurality of first electrodes are formed on the substrate, and a plurality of second electrodes are formed on the multilayer structure;
each nanowire extends between a first electrode and a second electrode;
each first electrode is connected to a first addressing unit, configured to select at least one first electrode;
each second electrode is connected to a second addressing unit, configured to select at least one second electrode;
such that the detection circuit detects a detection signal induced by each nanowire extending between the selected first electrode and the selected second electrode.

13. The device according to claim 12, wherein:
the first addressing unit is configured to successively select a plurality of first electrodes;
the second addressing unit is configured to successively select a plurality of second electrodes.

14. The device according to claim 12, wherein:
the substrate and the multilayer structure extend parallel to a longitudinal axis and to a lateral axis, the lateral axis and the longitudinal axis being secant to each other, in a plane secant to the transverse axis;
each first electrode is placed parallel to the longitudinal axis;
each second electrode is placed parallel to the lateral axis.

15. The device according to claim 12, wherein:
the functionalization surface is functionalized by a biological detection probe, the biological detection probe being configured to selectively capture a biomolecule of interest;
such that capture of the biomolecule of interest leads to a variation in the fluorescence light detected by at least one nanowire when the functionalization surface is illuminated with excitation light to excite a fluorescent label grafted to the biomolecule of interest or to the biological detection probe.

16. The device according to claim 15, wherein the biological detection probe is a molecular beacon.

17. The device according to claim 12, wherein:
the functionalization surface is segmented into a plurality of segments;
each segment of the functionalization surface lies facing at least one nanowire;
various segments of the functionalization surface are functionalized by biological detection probes configured to selectively capture various biomolecules of interest, respectively.

18. A method for detecting at least one biomolecule of interest using a device according to claim 1, the functionalization surface being functionalized beforehand with a biological detection probe configured to selectively capture the biomolecule of interest, the device comprising:
a) placing a sample, liable to contain the biomolecule of interest, in contact with the functionalization surface;
b) connecting the detection circuit to terminals of the first electrode and of the second electrode;
c) exposing the functionalization surface to excitation light, in an excitation spectral band of a fluorescent label, the fluorescent label being grafted to the biological detection probe or to the biomolecule of interest, the fluorescent label being configured to emit fluorescent light, in the detection spectral band, when it is illuminated by the excitation light;
d) depending on the detection signal, detecting a variation in fluorescence light detected by at least one nanowire, the variation in fluorescence light indicating capture of the biomolecule of interest by a biological detection probe.

19. The method according to claim 18, wherein:
the device further includes a plurality of nanowires, wherein:
a plurality of first electrodes are formed on the substrate, and a plurality of second electrodes are formed on the multilayer structure;
each nanowire extends between a first electrode and a second electrode;
each first electrode is connected to a first addressing unit, configured to select at least one first electrode;
each second electrode is connected to a second addressing unit, configured to select at least one second electrode;
such that the detection circuit detects a detection signal induced by each nanowire extending between the selected first electrode and the selected second electrode;
steps b) to d) are reiterated, modifying, between two successive iterations, the selected first electrode or the selected second electrode.
